(19) United States
(12) Patent Application Publication
(10) Pub. No.: US 2013/0252886 A1
(43) Pub. Date: Sep. 26, 2013

(54) METHODS OF DELAYING AND TREATING DIABETES

(71) Applicant: Brooke Ligon, Sullivan, ME (US)

(72) Inventor: Brooke Ligon, Sullivan, ME (US)

(21) Appl. No.: 13/880,907

(22) PCT Filed: Oct. 21, 2011
(86) PCT No.: PCT/US11/57253

§ 371 Date: Apr. 22, 2013

Related U.S. Application Data
(60) Provisional application No. 61/405,898, filed on Oct. 22, 2010.

(51) Int. Cl. A61K 31/197; A61K 38/28

(52) U.S. Cl. CPC A61K 31/197; A61K 38/28
USPC 514/6.5; 514/561

(57) ABSTRACT

The present invention provides methods of treating hyperglycemia and delaying the onset of diabetes, comprising administering to a mammal in need thereof a therapeutically effective amount of a GABA receptor agonist at or near a time of an exogenous insulin administration or a meal.

### RELATED APPLICATIONS

This application claims the benefit of U.S. Provisional Application No. 61/405,898, filed on Oct. 22, 2010, the entire teachings of which are hereby incorporated by reference.

### BACKGROUND OF THE INVENTION

Diabetes mellitus, often referred to simply as “diabetes,” is a condition in which a person has a high blood (plasma) level of the sugar glucose. This abnormally high level of glucose is generally either because the person's pancreas does not produce sufficient insulin or because cells do not properly respond to the insulin that is produced. Insulin is a hormone that enables cells to absorb glucose which is used to produce energy.

Although there are many types of diabetes, the two most common types are called Type 1 diabetes and Type 2 diabetes. In Type 1 diabetes, a person's pancreas fails to produce sufficient insulin and the person is usually reliant upon exogenous insulin. Type 2 can result from a number of etiologies, including insulin resistance, a condition in which cells fail to respond to insulin properly, and is sometimes combined with insulin deficiency.

Currently, there are millions of diabetic people in the world and many others in a pre-diabetic state. On top of that, the incidence of diabetes is significantly increasing. Because of this, there has been and currently is an extremely large amount of research directed to better understand the causes of diabetes and to develop new and better treatments. However, no currently available treatments are entirely satisfactory and there is a need for new efficacious treatments for treating people with diabetes, and for delaying or preventing the onset of diabetes to susceptible persons.

### SUMMARY OF THE INVENTION

The present invention relates to treating hyperglycemia in a mammal, particularly hyperglycemia caused by diabetes. In one embodiment, this invention is a method of treating hyperglycemia in a mammal in need thereof. This method comprises periodically administering to the mammal a therapeutically effective amount of a γ-aminobutyric acid (GABA) receptor agonist at or near a time of an exogenous insulin administration or at or near a time of a meal. In certain embodiments, the GABA receptor agonist is GABA.

In certain embodiments the therapeutically effective amount of the GABA receptor agonist comprises from about 30 mg/kg to about 120 mg/kg.

In another embodiment, the method comprises administering the GABA receptor agonist once, at least once, twice, at least twice, three times, at least three times, four times, or at least four times per day.

In another embodiment, the mammal is a human. In another embodiment, the hyperglycemia is caused by diabetes mellitus. In another embodiment, the method comprises administering a therapeutically effective amount of a pharmaceutical composition of a GABA receptor agonist. In another embodiment, the method comprises administering a therapeutically effective amount of a pharmaceutically acceptable carrier of a GABA receptor agonist. In yet another embodiment, the method comprises periodically administering a therapeutically effective amount of a pharmaceutically acceptable salt of a GABA receptor agonist to a mammal to treat hyperglycemia caused by diabetes.

In another embodiment, the method relates to delaying the onset of diabetes in a mammal subject to developing diabetes. This method comprises periodically administering an effective amount of a GABA receptor agonist at or near ingestion of meals by said mammal. In one embodiment, the GABA receptor agonist comprises GABA. In another embodiment, the mammal is a human. In certain embodiments, the effective amount of the GABA receptor agonist is an amount from about 30 mg/kg to about 120 mg/kg.

In another embodiment, the method relates to reducing the insulin dosage in the treatment of diabetes in a mammal. The method comprises co-administering an effective insulin-reducing amount of a GABA receptor agonist at or near the periodic administrations of insulin.

In another embodiment, the method relates to maintaining lean body mass in a mammal with diabetes comprising periodically administering an effective amount of a GABA receptor agonist at or near ingestion of meals. In another embodiment, the method relates to maintaining glucose homeostasis in a mammal in need thereof comprising periodically administering an effective amount of a GABA receptor agonist at or near ingestion of meals or an exogenous insulin administration. In another embodiment, the method relates to decreasing exogenous insulin required to maintain glucose homeostasis comprising periodically administering an effective amount of a GABA receptor agonist at or near ingestion of meals.

The advantages of the present invention include treatment methods to delay the onset of diabetes in mammals prone to developing diabetes, reducing the insulin requirements in a mammal being treated for diabetes, and maintaining lean body mass in mammals with diabetes using a GABA receptor agonist. Other advantages include methods to treat hyperglycemia, particularly hyperglycemia caused by diabetes, methods for maintaining glucose homeostasis using a GABA receptor agonist, and methods for preventing secondary complications caused by hyperglycemia.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a graph of the average fasting blood glucose in female NOD mice undergoing a variety of treatment regimens before becoming diabetic.

FIG. 2 is a graph of the average insulin dose for diabetic female NOD mice over the course of treatment with 60 mg/kg GABA.

FIG. 3 is a graph of the average weights of female NOD mice from each treatment group during a treatment with 30 mg/kg GABA.

FIG. 4 is a graph of the average weights of mice from each treatment group from 12 weeks to 32 weeks of age. Some mice within these groups are diabetic and on the intervention protocol of 60 mg/kg GABA.

FIG. 5 is a graph of the change in average insulin requirement of diabetic mice over 6 weeks on 60 mg/kg GABA treatment.

FIG. 6 is a graph of the mean insulin requirement of three diabetic NOD mice whose insulin dependency decreased to 0 units on 60 mg/kg GABA compared to control NOD mice.

FIG. 7 is a graph of the fasting blood glucose in six individual insulin-dependent canines treated with 60 mg/kg GABA beginning on day 60.

FIG. 8 is a graph of the mean fasting blood glucose in 5 dogs before and during 60 mg/kg GABA treatment.

FIG. 9 is a bar graph of the average plasma fructosamine (μmol/L) in six canines during pretreatment (days 0-59) versus during 30 mg/kg GABA treatment (days 60-120).

FIG. 10 is a graph of the average plasma fructosamine (μmol/L) in six canines during pretreatment (days 0-59) and during 30 mg/kg GABA treatment (days 60-120).

FIG. 11A is a graph of the mean insulin requirement in control NOD mice.

FIG. 11B is a graph of the mean insulin requirement in NOD mice treated with 60-90 mg/kg GABA.

FIG. 11C is a graph combining the data as shown in FIGS. 11A and 11B.

FIG. 12A is a graph of the mean blood glucose level in control NOD mice.

FIG. 12B is a graph of the mean blood glucose level in NOD mice treated with 60-90 mg/kg GABA.

FIG. 12C is a graph combining the data as shown in FIGS. 12A and 12B.

FIG. 13A is a graph of the average weekly insulin dose for a single NOD mouse that was completely weaned off of insulin.

FIG. 13B is a graph of the average weekly fasting blood glucose level in the same NOD mouse as in FIG. 13A.

FIG. 14 is a histology section from the pancreas of the NOD mouse in FIGS. 13A and B at 64 weeks.

FIG. 15 is a histology section from a NOD mouse pancreas that received 2× (60 mg/ml) GABA and insulin treatment until the time of sacrifice at 65 weeks of age.

FIG. 16 is a histology section from a normal, non-diabetic mouse pancreas.

FIG. 17 is a panel of histology sections from a control NOD mouse, not treated with GABA.

FIG. 18 is a graph of the mean fasting blood glucose (mg/dl) and insulin dosage (units) in 7 dogs measured over the study period.

FIG. 19 is a graph of the average C-peptide measurement (ng/ml/kg) in 7 dogs at various stages of treatment during the study period measured at 0, 60, 90, 120 and 240 minutes after feeding.

FIG. 20A is a graph of the mean GABA plasma concentration (ng/ml) in dogs at various stages of GABA treatment measured during the study period by liquid chromatography/mass spectrometry.

FIG. 20B is a graph of the mean GABA plasma concentration (ng/ml) in dogs at various stages of GABA treatment during the study period measured by liquid chromatography/mass spectrometry.

### DETAILED DESCRIPTION OF THE INVENTION

As used in the description of this invention, the terms set forth below have the following definitions.

The present invention is related to methods of treating hyperglycemia and delaying the onset of diabetes. The term “hyperglycemia” as used herein, means high blood sugar. In some instances, hyperglycemia is arbitrarily defined for a particular embodiment. One of ordinary skill in the art would readily recognize definitions, including that of hyperglycemia, are subject to change over time and the latest definitions and standards disclosed by organizations such as the World Health Organization and the American Diabetes Association can be used to define hyperglycemia as provided in the present invention.

The term “hyperglycemia” is also intended to include those individuals with chronic hyperglycemia, hyperinsulinemia, impaired glucose homeostasis or tolerance, insulin resistance, and diabetes. Plasma glucose levels in hyperglycemic individuals include, for example, glucose concentrations greater than normal as determined by reliable diagnostic indicators. Such hyperglycemic individuals are at risk or predisposed to developing overt clinical symptoms of diabetes mellitus.

The terms “plasma glucose” and “plasma glucose level” are sometimes referred to as “blood glucose” and “blood glucose level.” One of ordinary skill in the art readily recognizes glucose levels are measured in the intravascular fluid part of the extracellular fluid.

As used herein, the term “diabetes” is intended to mean all diabetic conditions, including, without limitation, diabetes mellitus, genetic causes of diabetes (e.g., maturity onset diabetes of the young (MODY)), type 1 diabetes, type 2 diabetes, and gestational diabetes. The term “diabetes” also refers to the chronic disease characterized by relative or absolute deficiency of insulin that results in hyperglycemia. Type 1 diabetes is also referred to as insulin dependent diabetes mellitus (IDDM) and also includes, for example, juvenile-onset diabetes mellitus. Type 1 is primarily due to the destruction of pancreatic β-cells. Type 2 diabetes mellitus is also known as non-insulin dependent diabetes mellitus (NIDDM) and is characterized, in part, by impaired insulin release following a meal. Insulin resistance can also be a factor leading to the occurrence of type 2 diabetes mellitus. Genetic causes of diabetes are due to mutations which interfere with the function and regulation of pancreatic β-cells.

Diabetes is characterized in humans as a fasting plasma glucose level greater than or equal to about 126 mg/dl. Diabetes can also be characterized as a plasma glucose level greater than or equal to about 180 mg/dl as assessed at about 2 hours following an oral ingestion of a glucose load of about 75 grams or following a meal. In certain embodiments of the present invention, diabetes is arbitrarily defined as a fasting plasma glucose ≧200 mg/dl. One of ordinary skill in the art would appreciate the various ways to diagnose diabetes in a variety of mammals. Further, the characteristics used in identifying and diagnosing diabetes are subject to change and the latest standards, such as those disclosed by the World Health Organization, can be used to define diabetes as provided in the present invention.

In certain embodiments, the method is related to delaying the onset of diabetes. In another embodiment, the method is related to reducing the insulin dosage in the treatment of diabetes. In another embodiment, the method is related to maintaining lean body mass in a mammal with diabetes. In one embodiment, the method is related to treating hyperglycemia in a mammal. In one embodiment, the method is related to treating diabetes. In another embodiment, the method is related to maintaining glucose homeostasis.

As used herein, the terms “diabetic complication” and “secondary complication” refer to medical or clinical problems that can occur in patients diagnosed with diabetes. These include, and are not limited to, metabolic disorders (e.g., ketoacidosis, gout, hypercholesterolemia, hypoglycemia, non-ketotic hyperglycemic coma), urologic disorders, dermatologic conditions (e.g., diabetic dermopathy, necrobiosis lipoidica diabeticorum, bullosis diabeticorum, eruptive xanthomatosis, allergic skin reactions, digital scleroris, disseminated granuloma annulare, and acanthosis nigricans), gum disease, retinopathy (e.g., glaucoma, cataracts, non-proliferative retinopathy, diabetic macular edema, vitreous hemorrhage with retina detachment, proliferative retinopathy with retinal vascular miscoaneurysm, neovascularization hemorrhage, retinal detachment, rebeosa iridis), nephropathy (e.g. acute renal failure, chronic renal failure), neuropathy (e.g., systemic neuropathy, distal systemic polyneuropathy, proximal neuropathy, anterior ischemic optic neuropathy, femoral neuropathy, neuropathic anthropathy, cranial neuropathy, autonomic neuropathy, compression neuropathy, diabetic mononeuropathy, neuropathic pain, thoracic radiculopathy, and diabetic amyotrophy), infections (e.g., bacterial infections, fungal infections, nosocomial infections), erectile dysfunction, gastrointestinal disorders (e.g., gastroparesis, duiabetic fatty liver, diabetic steatonecrosis) and cardiovascular diseases and related disorders (e.g., hypertension, heart disease, heart attack, CHF, stroke, vascular disease, ischemia).

In one embodiment, this invention is related to treating hyperglycemia in a mammal in need thereof comprising periodically administering to the mammal a therapeutically effective amount of a γ-aminobutyric acid (GABA) receptor agonist at or near a time of an exogenous insulin administration. In one embodiment, the method comprises periodically administering to the mammal a therapeutically effective amount of a pharmaceutical composition of a GABA receptor agonist. In one embodiment, the method comprises administering the GABA receptor agonist as a pharmaceutically acceptable salt. In another embodiment, the method comprises administering the GABA receptor agonist in a pharmaceutically acceptable carrier.

In one embodiment, the hyperglycemia is caused by diabetes mellitus. In another embodiment, the diabetes mellitus is Type 1 diabetes. In another embodiment, the diabetes mellitus is Type 2 diabetes. In one embodiment, the hyperglycemia is caused by gestational diabetes. In another embodiment, the hyperglycemia is caused by impaired glucose tolerance. In another embodiment, the hyperglycemia is caused by insulin resistance. In another embodiment, the hyperglycemia is caused by medications. In another embodiment, the hyperglycemia is caused by an infection.

In one embodiment, the method comprises administering to a mammal a therapeutically effective amount of a GABA receptor agonist preventing the onset of a secondary complication caused by diabetes mellitus. In another embodiment, the method comprises administering to a mammal a therapeutically effective amount of a GABA receptor agonist reducing the risk of the mammal developing a secondary complication as a result of having diabetes.

In one embodiment, the method comprises administering the GABA receptor agonist at or near a time or ingestion of a meal (i.e., periprandial). The terms “at or near a time of a meal” or “at or near ingestion of a meal” as used herein, means the GABA receptor agonist is administered shortly before, during, or shortly after the meal is consumed. As used herein, the term “meal” is intended to include any time solid, semi-solid or liquid food is consumed. This includes, and is not limited to, breakfast, lunch, and dinner. This can occur in any setting and at any time. For example, this includes meals that are administered or ingested in a hospital or hospital-like setting (e.g., a nursing home). In these instances, a meal can be delivered via a feeding tube used for enteral feeding. For example, a nasogastric (NG) tube or a percutaneous endoscopic gastrostomy (PEG) tube can be used in these instances. Enteral feeding through an NG or a PEG tube can be used for continuous or bolus feedings. In another embodiment of the present invention, the method comprises administering the GABA agonist at a time a meal is consumed.

In another embodiment, the method comprises administering the GABA agonist at or near a time of an exogenous insulin administration. The term “at or near a time of an exogenous insulin administration” as used herein, means the GABA agonist is administered shortly before, during, or shortly after exogenous insulin administration. The term “exogenous insulin administration” as used herein, means receiving insulin from other than the pancreatic beta cells within the person's body.

Administration of the GABA receptor agonist can be periodic in nature and occur at various times during the day. In certain embodiments, the GABA receptor agonist is administered at or near exogenous insulin administration. In one embodiment of the present invention, the GABA receptor agonist is administered less than about two hours prior to administration of exogenous insulin. In one embodiment of the present invention, the GABA receptor agonist is administered less than about one hour prior to administration of exogenous insulin. In another embodiment, the GABA receptor agonist is administered less than about 30 minutes prior to administration of exogenous insulin. In another embodiment, the GABA receptor agonist is administered less than about 15 minutes prior to administration of exogenous insulin. In another embodiment, the GABA receptor agonist is administered less than about 5 minutes prior to administration of exogenous insulin. In other embodiments of the present invention, the GABA receptor agonist is administered less than about two hours after administration of exogenous insulin. In other embodiments of the present invention, the GABA receptor agonist is administered less than about one hour after administration of exogenous insulin. In one embodiment, the GABA receptor agonist is administered less than about 30 minutes after administration of exogenous insulin. In another embodiment, the GABA receptor agonist is administered less than about 15 minutes after administration of exogenous insulin. In another embodiment, the GABA receptor agonist is administered less than about 5 minutes after administration of exogenous insulin. In still another embodiment, the GABA receptor agonist and exogenous insulin are administered at about the same time. The administration of the GABA receptor agonist and exogenous insulin can require varying dosages for a given mammal and could result in dose-to-dose variability. In one embodiment, the amount of exogenous insulin required to maintain a normal blood glucose level in the mammal decreases over time, when administered with a GABA receptor agonist. In another embodiment, the amount of exogenous insulin administered eventually decreases to zero, when administered with a GABA receptor agonist.

Periodic administration of the GABA receptor agonist at or near meals can occur at various times during the day. In one embodiment of the present invention, the GABA receptor agonist is administered less than about two hours before a meal. In one embodiment of the present invention, the GABA receptor agonist is administered less than about one hour before a meal. In another embodiment, the GABA receptor agonist is administered less than about 30 minutes before a meal. In another embodiment, the GABA receptor agonist is administered less than about 15 minutes before a meal. In another embodiment, the GABA receptor agonist is administered less than about 5 minutes before a meal. In other embodiments of the present invention, the GABA receptor agonist is administered less than about two hours after a meal. In other embodiments of the present invention, the GABA receptor agonist is administered less than about one hour after a meal. In one embodiment, the GABA receptor agonist is administered less than about 30 minutes after a meal. In another embodiment, the GABA receptor agonist is administered less than about 15 minutes after a meal. In another embodiment, the GABA receptor agonist is administered less than about 5 minutes after a meal. In yet another embodiment, the GABA receptor agonist is administered at approximately the same time with a meal.

The GABA receptor agonist can be used at doses appropriate for other conditions for which GABA agonists are known to be useful. The typical daily dose of the active substance varies within a wide range and will depend on various factors, such as, the individual requirement of each mammal and the route of administration. The term “mg/kg,” as used herein means “mg” of GABA receptor agonist per “kg” of body weight. In certain embodiments, the therapeutically effective amount of the GABA receptor agonist comprises from about 1 to about 300 mg per day per kg body weight. In one embodiment, the therapeutically effective amount of the GABA receptor agonist comprises from about 7.5 to about 180 mg/kg. In another embodiment, the therapeutically effective amount of the GABA receptor agonist comprises approximately from about 30 to about 120 mg/kg. In certain embodiments, the therapeutically effective amount of the GABA receptor agonist comprises about 7.5 mg/kg. In certain embodiments, the therapeutically effective amount of the GABA receptor agonist comprises about 10 mg/kg. In certain embodiments, the therapeutically effective amount of the GABA receptor agonist comprises about 15 mg/kg. In certain embodiments, the therapeutically effective amount of the GABA receptor agonist comprises about 30 mg/kg. In certain embodiments, the therapeutically effective amount of the GABA receptor agonist comprises about 60 mg/kg. In certain embodiments, the therapeutically effective amount of the GABA receptor agonist comprises about 90 mg/kg. In certain embodiments, the therapeutically effective amount of the GABA receptor agonist comprises about 120 mg/kg. In certain embodiments, the therapeutically effective amount of the GABA receptor agonist comprises about 180 mg/kg. In certain embodiments, the therapeutically effective amount of the GABA receptor agonist comprises about 270 mg/kg.

In one embodiment, the GABA receptor agonist is a GABA_{A} and a GABA_{B} receptor agonist. In another embodiment, the GABA receptor agonist is γ-aminobutyric acid (GABA). In certain embodiments, the therapeutically effective amount of GABA comprises from about 10 to about 100 mg per kg body weight for each periodic administration. In one embodiment, the therapeutically effective amount of GABA comprises from about 20 to about 80 mg/kg. In another embodiment, the therapeutically effective amount of GABA comprises approximately from about 30 to about 60 mg/kg. In certain embodiments, the therapeutically effective amount of GABA comprises about 10 mg/kg. In certain embodiments, the therapeutically effective amount of GABA comprises about 20 mg/kg. In certain embodiments, the therapeutically effective amount of GABA comprises about 30 mg/kg. In certain embodiments, the therapeutically effective amount of GABA comprises about 60 mg/kg. In certain embodiments, the therapeutically effective amount of GABA comprises about 75 mg/kg. In certain embodiments, the therapeutically effective amount of GABA comprises about 80 mg/kg. In certain embodiments, the therapeutically effective amount of GABA comprises about 100 mg/kg.

In another embodiment, the GABA receptor agonist is a partial GABA_{A} receptor agonist and a partial GABA_{B} receptor agonist. In another embodiment, the GABA receptor agonist does not significantly cross the blood-brain barrier when administered at effective dosages for the treatment of diabetes. In another embodiment, the GABA receptor agonist is a GABA_{A} receptor agonist or a GABA_{B} receptor agonist.

Examples of GABA receptor agonists with complete or partial affinity to both GABA_{A} and GABA_{B} receptors are: progabide and its metabolites (e.g. gabamide).

Examples of GABA receptor agonists with complete or partial affinity to GABA_{A} receptors are: muscimol, isoguvacine, 4,5,6,7-tetrahydroisoxazolo[5,4-c]pyridin-3-ol: imidazole-4-ethanoic acid, (RS) 2-amino-2-thiazoline-4-ethanoic acid, (z)-3-[(aminoiminomethyl)-thio]prop-2-enoic acid, (1S,3S)-3-aminocyclopentane-1-carboxylic acid, Thio-THIP, Isonipecotic acid, SL75102, dihydromuscimol (S-DHM), 1,2,3,6-tetrahydropyridine-4-sulfonic acid, Piperidine-4-suflonic acid, Calcium N-acetylhomotaurinate, homotaurine, trans-aminocyclopentane-3-carboxylic acid, trans-amino-4-crotonic acid, imidazole acetic acid, β-guanidino-propionic acid, homohypotaurine, 3-aminopropanesulfonic acid, kojic amine, and homo-β-proline.

Examples of GABA receptor agonists with complete or partial affinity to GABA_{B} receptors are: 4-amino-3-(4-chlorophenyl)butanoic acid (baclofen), 4-aminobutanoic acid, 4-amino-3-phenylbutanoic acid, 4-amino-3-hydroxybutanoic acid, 4-amino-3-(4-chlorophenyl)-3-hydroxyphenylbutanoic acid, 4-amino-3-(thien-2-yl)butanoic acid, 4-amino-3-(5-chlorothien-2-yl)butanoic acid, 4-amino-3-(5-bromothien-2-yl)butanoic acid, 4-amino-3-(5-methylthien-2-yl)butanoic acid, 4-amino-3-(2-imidazolyl)butanoic acid, 4-guanidino-3-(4-chlorophenyl)butanoic acid, 3-amino-2-(4-chlorophenyl)-1-nitropropane, (3-aminopropyl)phosphonous acid, (4-aminobut-2-yl)phosphonous acid, (3-amino-2-methylpropyl)phosphonous acid, (3-aminobutyl)phosphonous acid, (3-amino-2-(4-chlorophenyl)propyl)phosphonous acid, (3-amino-2-(4-chlorophenyl)-2-hydroxypropyl)phosphonous acid, (3-amino-2-(4-fluorophenyl)propyl)phosphonous acid, (3-amino-2-phenylpropyl)phosphonous acid, (3-amino-2-hydroxypropyl)phosphonous acid, (E)-(3-aminopropen-1-yl)phosphonous acid, (3-amino-2-cyclohexylpropyl)phosphonous acid, (3-amino-2-benzylpropyl)phosphonous acid, [3-amino-2-(4-methylphenyl)propyl]phosphonous acid, [3-amino-2-(4-trifluoromethylphenyl)propyl]phosphonous acid, [3-amino-2-(4-methoxyphenyl)propyl]phosphonous acid, [3-amino-2-(4-chlorophenyl)-2-hydroxypropyl]phosphonous acid, (3-aminopropyl)methylphosphinic acid, (3-amino-2-hydroxypropyl)methylphosphinic acid, (3-aminopropyl)(difluoromethyl)phosphinic acid, (4-aminobut-2-yl)methylphosphinic acid, (3-amino-1-hydroxypropyl)methylphosphinic acid, (3-amino-2-hydroxypropyl)(difluoromethyl)phosphinic acid, (E)-(3-aminopropen-1-yl)methylphosphinic acid, (3-amino-2-oxo-propyl)methyl phosphinic acid, (3-aminopropyl)hydroxymethylphosphinic acid, (5-aminopent-3-yl)methylphosphinic acid, (4-amino-1,1,1-trifluorobut-2-yl)methylphosphinic acid, (3-amino-2-(4-chlorophenyl)propyl)sulfinic acid, and 3-aminopropylsulfinic acid.

Examples of GABA_{B} receptor allosteric modulators are: N,N′-dicylcopentyl-2-methylsulfanyl-5-nitropyrimidine-4,6-diamine and analogs, 2,6-di-tert-butyl-4-(3-hydroxy-2,2-dimethylpropyl)-phenol and analogs, 5,7-bis(1,1-dimethylethyl)-3-hydroxy-3(trifluoromethyl)-2(3H)-benzofuranone, N-[(1R,2R,4S)-bicyclo[2.2.1]hept-2-yl]-2-methyl-5-[4-(trifluoromethyl)phenyl]-4-pyrimidinamine, and 2,6-di-tert-butyl-4-(3-hydroxy-2-spiropentylpropyl)-phenol.

Examples of GABA_{A} receptor allosteric modulators are: diazepam derivatives, valium, ativan, triazolopyridazine derivatives, pyrazolo-pyridine derivatives, nicotinic carboxamide compounds, neuroactive steroids, such as androstane and pregnane derivatives, triazolophthalazine derivatives, tricyclic pyrazolo-pyridazinone analogs, barbiturates and fenamates.

In one embodiment the mammal is a human. In another embodiment, the mammal is a non-human primate. In another embodiment, the mammal is a canine. In another embodiment, the mammal is a feline.

In certain embodiments, the method comprises administering the GABA receptor agonist once (QD), at least once, twice (BID), at least twice, three times (TID), at least three times, four times (QID), or at least four times per day. In one embodiment, the method comprises administering the GABA receptor agonist at least once per day. In another embodiment, the method comprises administering the GABA receptor agonist at least twice per day. In another embodiment, the method comprises administering the GABA receptor agonist at least three times per day. In another embodiment, the method comprises administering the GABA receptor agonist at least four times per day. In another embodiment, the method comprises administering the GABA receptor agonist as many times that is necessary to maintain a normal blood glucose level. In this instance, the GABA receptor agonist dosing is specifically tailored to a specific mammal and can vary from dose to dose and from day to day. Similar to an insulin sliding scale, the dose of a GABA receptor agonist, in one embodiment, can be dependant upon a mammal's blood glucose. One of ordinary skill in the art would readily appreciate the dosing variability of the GABA receptor agonist to maintain a blood glucose level within the normal range.

In one embodiment, the compounds of the invention can be present in the form of pharmaceutically acceptable compositions. In another embodiment, the compounds of the invention can be present in the form of pharmaceutically acceptable salts. For use in medicines, the salts of the compounds of the invention refer to non-toxic pharmaceutically acceptable salts.

The pharmaceutically acceptable salts of the GABA receptor agonists include acid addition salts and base addition salts. The term “pharmaceutically acceptable salts” embraces salts commonly used to form alkali metal salts and to form addition salts of free acids or free bases. The nature of the salt is not critical, provided that it is pharmaceutically acceptable.

Suitable pharmaceutically acceptable acid addition salts of the GABA receptor agonists can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids, examples of which are formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, and galacturonic acid. Pharmaceutically acceptable acidic/anionic salts also include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphospate, polygalacturonate, salicylate, stearate, subacetate, succinate, sulfate, hydrogensulfate, tannate, tartrate, teoclate, tosylate, and triethiodide salts.

Suitable pharmaceutically acceptable base addition salts of the GABA receptor agonists include, but are not limited to, metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine. All of these salts can be prepared by conventional means from the corresponding compound represented by the disclosed compound by treating, for example, the disclosed compounds with the appropriate acid or base. Pharmaceutically acceptable basic/cationic salts also include, the diethanolamine, ammonium, ethanolamine, piperazine and triethanolamine salts.

In an embodiment, the pharmaceutically acceptable salt comprises a monovalent cation or a divalent cation. In a particular embodiment, the pharmaceutically acceptable salt is a lysine salt.

In another embodiment, the monovalent cation is a monovalent metal cation and the divalent cation is a divalent metal cation. In a particular embodiment, the monovalent metal cation is a sodium cation.

The pharmaceutical compositions disclosed herein are prepared in accordance with standard procedures and are administered at dosages that are selected to reduce, prevent, or eliminate, or to slow or halt the progression of, the condition being treated (See, e.g., Remington's Pharmaceutical Sciences, Mack Publishing Company, Easton, Pa., and Goodman and Gilman's The Pharmaceutical Basis of Therapeutics, McGraw-Hill, New York, N.Y., the contents of which are incorporated herein by reference, for a general description of the methods for administering various agents for human therapy). The compositions of a compound represented by the disclosed compounds can be delivered using controlled or sustained-release delivery systems (e.g., capsules, biodegradable matrices). Exemplary delayed-release delivery systems for drug delivery that would be suitable for administration of the compositions of the disclosed compounds are described in U.S. Pat. Nos. 5,990,092 (issued to Walsh); 5,039,660 (issued to Leonard); 4,452,775 (issued to Kent); and 3,854,480 (issued to Zaffaroni), the entire teachings of which are incorporated herein by reference.

For preparing pharmaceutical compositions from the GABA receptor agonists of the present invention, pharmaceutically acceptable carriers can either be solid or liquid. Solid form preparations include powders, tablets, pills, capsules, cachets, suppositories, and dispersible granules. For example, the compounds of the present invention can be in powder form for reconstitution at the time of delivery. A solid carrier can be one or more substances which can also act as diluents, flavoring agents, solubilizers, lubricants, suspending agents, binders, preservatives, tablet disintegrating agents, or an encapsulating material. In powders, the carrier is a finely divided solid which is in a mixture with the finely divided active ingredient.

In tablets, the active ingredient is mixed with the carrier having the necessary binding properties in suitable proportions and compacted in the shape and size desired.

The powders and tablets preferably contain from about one to about seventy percent of the active ingredient. Suitable carriers are magnesium carbonate, magnesium stearate, talc, sugar, lactose, pectin, dextrin, starch, gelatin, tragacanth, methylcellulose, sodium caboxymethylcellulose, a low-melting wax, cocoa butter, and the like. Tablets, powders, cachets, lozenges, fast-melt strips, capsules and pills can be used as solid dosage forms containing the active ingredient suitable for oral administration.

Liquid form preparations include solutions, suspensions, retention enemas, and emulsions, for example, water or water propylene glycol solutions. For parenteral injection, liquid preparations can be formulated in solution in aqueous polyethylene glycol solution.

Aqueous solutions suitable for oral administration can be prepared by dissolving the active ingredient in water and adding suitable colorants, flavors, stabilizing agents, and thickening agents as desired. Aqueous suspensions for oral administration can be prepared by dispersing the finely divided active ingredient in water with viscous material, such as natural or synthetic gums, resins, methylcellulose, sodium carboxymethylcellulose, and other well-known suspending agents.

The pharmaceutical composition is preferably in unit dosage form. In such form, the composition is subdivided into unit doses containing appropriate quantities of the active ingredient. The unit dosage form can be a packaged preparation, the package containing discrete quantities of, for example, tablets, powders, and capsules in vials or ampules. Also, the unit dosage form can be a tablet, cachet, capsule, or lozenge itself, or it can be the appropriate amount of any of these in packaged form. The dosages can be varied depending upon the requirements of the patient, the severity of the condition being treated, the compound and the route of administration being employed. Determination of the proper dosage for a particular situation is within the skill in the art. Also, the pharmaceutical composition can contain, if desired, other compatible therapeutic agents.

In general, the methods for delivering the disclosed compounds and pharmaceutical compositions of the invention in vivo utilize art-recognized protocols for delivering the agent with the only substantial procedural modification being the substitution of the compounds represented by any one of the disclosed compounds for the drugs in the art-recognized protocols.

The compounds and compositions can, for example, be administered intravascularly, intramuscularly, subcutaneously, intraperitoneally, transmucosally, transdermally, orally or topically. One of ordinary skill in the art will recognize that the following dosage forms can comprise as the active ingredient, either compounds or a corresponding pharmaceutically acceptable salt of a compound of the present invention. One embodiment of the invention is oral administration of the compounds.

For oral administration, embodiments of the GABA receptor agonist and pharmaceutical compositions thereof include, but are not limited to, a tablet, capsule, suspension or liquid. The composition is preferably made in the form of a dosage unit containing a therapeutically effective amount of the active ingredient. Examples of dosage units are tablets and capsules. For therapeutic purposes, the tablets and capsules can contain, in addition to the active ingredient, conventional carriers such as binding agents, for example, acacia gum, gelatin, polyvinylpyrrolidone, sorbitol, or tragacanth; fillers, for example, calcium phosphate, glycine, lactose, maize-starch, sorbitol, or sucrose; lubricants, for example, magnesium stearate, polyethylene glycol, silica, or talc; disintegrants, for example potato starch, flavoring or coloring agents, or acceptable wetting agents. Oral liquid preparations generally in the form of aqueous or oily solutions, suspensions, emulsions, syrups or elixirs can contain conventional additives such as suspending agents, emulsifying agents, non-aqueous agents, preservatives, coloring agents and flavoring agents. Examples of additives for liquid preparations include acacia, almond oil, ethyl alcohol, fractionated coconut oil, gelatin, glucose syrup, glycerin, hydrogenated edible fats, lecithin, methyl cellulose, methyl or propyl para-hydroxybenzoate, propylene glycol, sorbitol, or sorbic acid.

Determining the dosage and route of administration for a particular composition in a mammal is well within the abilities of one of skill in the art. Preferably, the dosage does not cause or produces only minimal adverse side effects.

A therapeutically effective amount of a composition of the invention can be administered alone, or in combination with one or more other therapeutic agents. Suitable therapeutic agents that are useful for treating hyperglycemia, including hyperglycemia caused by diabetes, which can be administered in combination with a compound of the invention, include, but are not limited to sulfonylureas, biguanides, thiazolidinediones, α-glucosidase inhibitiors, meglitinides, dipeptidyl peptidase IV (DPP-IV) inhibitors, glucagon-like peptide-1 (GLP-1) and GLP-1 analogs. Suitable therapeutic agents that are useful for treating complications caused by diabetes include, but is not limited to, calcium channel blockers, beta blockers, nitroglycerin, aspirin, anti-inflammatory agents, natriuretic factors, vasodilators, thrombolytic and antithrombolic agents.

Thus, the GABA receptor agonists of the invention can be administered as part of a combination therapy (e.g., with each other, or with one or more other therapeutic agents). The compounds of the invention can be administered before, after or concurrently with one or more other therapeutic agents. In some embodiments, a compound of the invention and other therapeutic agent can be co-administered simultaneously (e.g., concurrently) as either separate formulations or as a joint formulation. Alternatively, the agents can be administered sequentially, as separate compositions, within an appropriate time frame, as determined by the skilled clinician (e.g., a time sufficient to allow an overlap of the pharmaceutical effects of the therapies). A compound of the invention and one or more other therapeutic agents can be administered in a single dose or in multiple doses, in an order and on a schedule suitable to achieve a desired therapeutic effect. Suitable dosages and regimens of administration can be determined by a clinician and are dependent on the agent(s) chosen, pharmaceutical formulation and route of administration, various patient factors and other considerations.

Typically, the efficacy of a pharmacological agent is directly related to its plasma concentration, wherein efficacy is reduced as plasma concentration falls. Thus, in standard multi-dosing regimens, a pharmacological agent is administered on a dosage schedule that is designed to maintain a pre-determined or optimal plasma concentration in the subject undergoing treatment. When the agent is administered at dosage intervals that are longer than the optimal interval(s), its plasma concentration can fall to undesirably low levels before the next dose is administered, with a concomitant decrease in efficacy.

### EXEMPLIFICATION

Non-obese diabetic (NOD) mice, genetically prone to develop autoimmune diabetes, serve as an excellent model for testing treatments for the analogous type 1 diabetes in humans. Normally, ninety to one hundred percent of female NOD mice, available, for example, from Jackson Laboratory, develop diabetes by 30 weeks of age. Two treatments were designed to study delaying the onset of diabetes and diabetes intervention in NOD mice. The first examined therapies in NOD mice beginning at 8 weeks of age to delay the onset of diabetes onset. The second examined therapies to treat diabetes in hyperglycemic NOD mice that had previously failed treatment to delay the onset of diabetes.

In the diabetes prevention/delayed onset study, mice were divided into five groups. The control group (Group 1) were treated only with carrier alone, applied to their fur (vegetable oil, 30 μl), and was consumed orally when grooming Group 2 was treated with 30 mg/kg GABA diluted to 50 mg/ml in water. The GABA solution was administered orally by pipette tip just prior to being allowed access to food and feeding. Group 3 received 0.4 units insulin subcutaneously just prior to consuming food. Group 4 received 30 mg/kg GABA (orally or “PO,” which stands for “per os”) and 0.4 units insulin subcutaneously just prior to consuming food. Group 5 was treated with 30 mg/kg GABA by pipette tip two hours before they were allowed to feed.

The results demonstrate that treatment with GABA just prior to feeding (Group 2) was the most successful prevention treatment. At 22 weeks of age, Group 2 was the only group with 100% of the mice (4/4 mice) alive and diabetes free. The timing of GABA administration in Group 2 was with a meal, at or near when endogenous insulin is secreted. At 24 weeks of age, the NOD mice in Group 2 (30 mg/kg GABA) and Group 4 (0.4 units insulin+30 mg/kg GABA) had average fasting blood glucose (mg/dl) levels significantly lower than other treatment groups.

Treatment with the identical dose of GABA (30 mg/kg) but under fasting conditions (Group 5), when endogenous insulin is not secreted, was less effective. In that group, 50% of the mice became diabetic at 22 weeks of age and had an average fasting blood glucose significantly higher at 24 weeks. This disparity of results, particularly in two groups that only differ by treatment time in relation to feeding, suggests insulin and GABA can work synergistically to maintain normal glucose metabolism.

Once the NOD mice became diabetic, they were shifted from the prevention/delayed onset regimen into the treatment protocol. This protocol involved treating the mice with 60 mg/kg GABA (PO BID). The first dose of GABA was given just after removing the food from the cage and a second dose was given approximately eight hours later, just before feeding. GABA was diluted in water at 50 mg/ml and administered orally by pipette. Two NOD mice were maintained on insulin when they became diabetic, to serve as controls.

The results of the treatment experiment showed that after the onset of diabetes, 60 mg/kg GABA was an effective treatment in lowering blood glucose levels and insulin requirements. Mice treated with 60 mg/kg GABA twice a day required significantly less insulin over time than those mice not receiving GABA. Furthermore, four insulin dependent diabetic mice became insulin independent after 60 mg/kg GABA treatment. In these mice, fasting and fed blood glucose was completely restored to normal after 60 mg/kg GABA was administered orally over approximately 11 weeks. These results provide evidence that either GABA is regenerating endogenous insulin production, improving insulin sensitivity, or both. The results also show that GABA treatment maintains lean body mass.

In summary, the data support the hypothesis that γ-aminobutyric acid works in concert with insulin to maintain blood glucose and body mass homeostasis. There is now evidence for the effective dose for delaying the onset of diabetes and treatment and intervention of diabetes, the timing of the effective dose, and the maintenance dosing requirements.

### Example 1

The first experiment examined whether GABA could prevent or delay the onset of diabetes. This experiment also tested whether GABA was more effective when insulin was available. Female NOD mice (Jackson Laboratory) were allowed to access food ad lib overnight. A 12 hour light-dark cycle was maintained and mice were housed in accordance with the NIH Animal Care and Use guidelines. Food was removed for eight hours during the day and the fasting blood glucose was tested daily at the end of the eight hour fast. Table 1 shows the average fasting blood glucose for each group.

TABLE 1
Average Fasting Blood Glucose: Non-obese diabetic mice on prevention Rx

| | 13 | 15 | 17 | 19 | 21 | 22 | 24 | 26 | |
|---|---|---|---|---|---|---|---|---|---|
| | weeks | weeks | weeks | weeks | weeks | weeks | weeks | weeks | |
| Group 1 | 100 | 96 | 113 | 148 | 111 | 159 | 251 | 283 | Group 1 |
| Control | | | | | | | | | Control |
| (n = 4) | | | | | | | | | (n = 4) |
| Group 2 | 80 | 111 | 119 | 127 | 138 | 173 | 152 | 253 | Group 2 |
| Gaba Only | | | | | | | | | Gaba Only |
| (n − 4) | | | | | | | | | (n − 4) |
| Group 3 | 82 | 114 | 162 | 134 | 150 | 188 | 195 | 260 | Group 3 |
| Insulin | | | | | | | | | Insulin |
| Only (n = 4) | | | | | | | | | Only (n = 3) |
| Group 4 | 96 | 111 | 156 | 136 | 135 | 157 | 155 | 202 | Group 4 |
| Insulin & | | | | | | | | | Insulin & |
| Gaba (n = 3) | | | | | | | | | Gaba (n = 1) |
| Group 5 | 86 | 97 | 100 | 172 | 239 | 266 | 265 | 226 | Group 5 |
| Fasting | | | | | | | | | Fasting |
| Gaba (n = 4) | | | | | | | | | Gaba (n = 3) |

Mice were placed in one of five treatment groups: (1) control, (2) 30 mg/kg GABA only, (3) 0.4 U Insulin only, (4) 0.4 U Insulin+30 mg/kg GABA, and (5) fasting 30 mg/kg GABA. The control group received vegetable oil to the fur without anything else. The GABA only group was given 30 mg/kg GABA in water orally (PO) just before having access to food (ad lib). The insulin only group was given 0.4 units insulin (SQ) just before feeding. Group 4 received 0.4 units of insulin and 30 mg/kg GABA PO just before feeding (ad lib). The fasting GABA group received 30 mg/kg GABA 2 hours prior to having access to food and feeding.

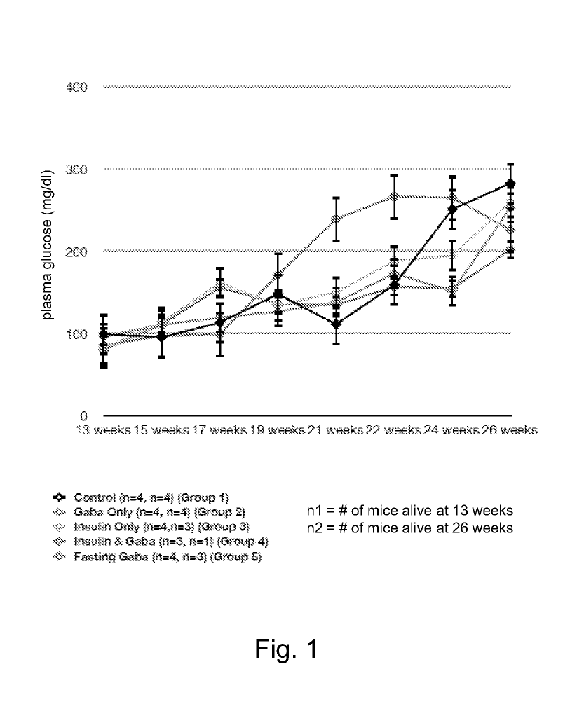

Diabetes was defined as a fasting blood glucose ≧200 mg/dl on three consecutive days. The average of the fasting blood glucose over the three days was included in the calculation of the average for each group in the prevention data (see Table 1 and FIG. 1).

FIG. 1 shows that diabetes onset was delayed the longest in Group 2, which received 30 mg/kg GABA just before the mice were allowed access to food and feeding. In Group 2, 100% (4/4) of the mice were diabetes free at 22 weeks. At 24 weeks the average fasting blood glucose for Group 2 was 152 mg/dl. Group 4 (0.4 U insulin+30 mg/kg GABA) had one diabetic mouse at 22 weeks and the group had an average fasting blood glucose of 155 mg/dl at 24 weeks. The change in number of mice in each group over the course of the study was due to ketoacidosis in one mouse and hypoglycemia in three others.

### Example 2

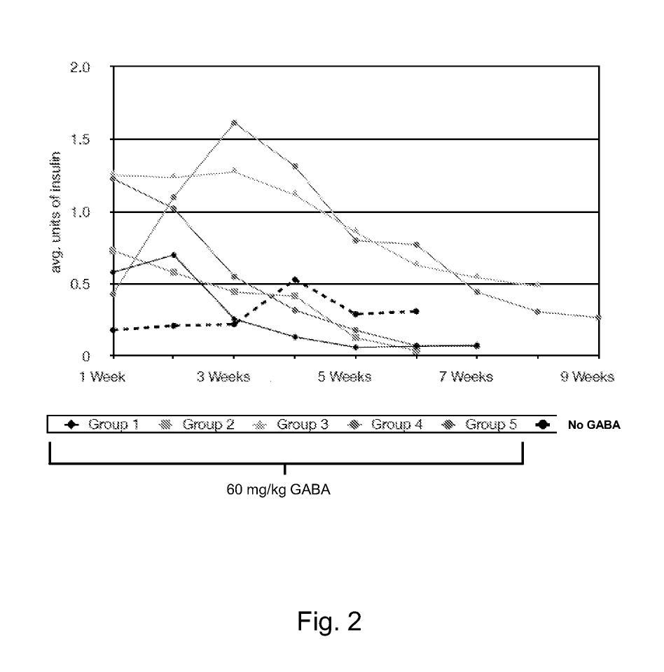

The female NOD mice previously described in Example 1 that reached a fasting blood glucose ≧200 mg/dl on three consecutive days were entered into the diabetes treatment protocol consisting of 60 mg/kg GABA given just prior to insulin administration twice a day (BID). The first dose of GABA was given just after removing the food from the cage and a second dose was given approximately eight hours later, just before feeding. The insulin dose administered was based on each mouse's blood glucose level. FIG. 2 shows the insulin dose required to maintain a fasting blood glucose ≦200 mg/dl declined markedly over the course of treatment with 60 mg/kg GABA. The one mouse not started on 60 mg/kg GABA (as depicted as ---- in FIG. 2) required steadily increasing doses of insulin to maintain a fasting blood glucose ≦200 mg/dl.

### Example 3

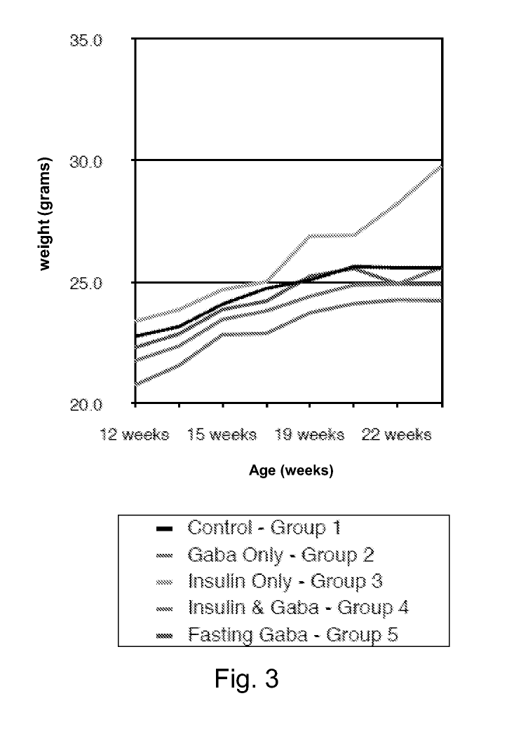

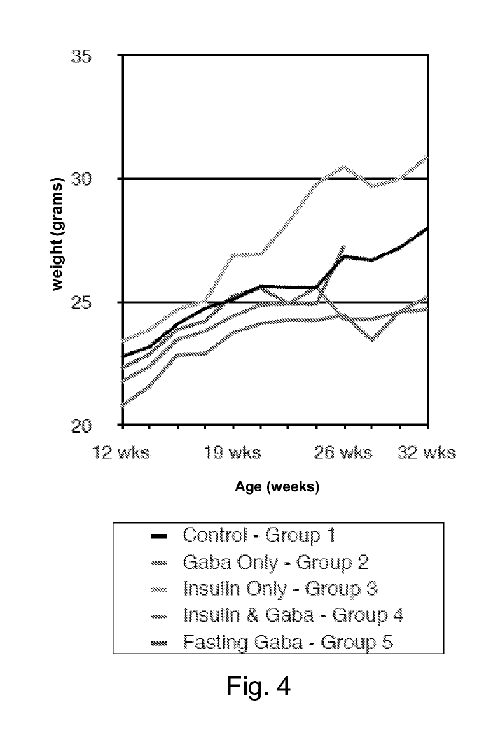

The GABA treatment (30 mg/kg) also affected lean body weight in NOD mice. FIGS. 3 and 4 show the average weight for the NOD mice in each group during the course of the experiment. The x-axis (age of mouse in weeks) corresponds to both the delayed onset and treatment regimens, depending on when the diagnosis of diabetes was made. Groups 2 and 4 maintained the lowest weights during this period showing that 30 mg/kg GABA or 30 mg/kg GABA plus insulin work to maintain lean body mass. Even after the mice became diabetic the weight trends were maintained as seen in FIG. 4 (average weights out to 32 weeks). The mice in Group 3 (0.4 U insulin only) increased body mass throughout the study.

As previously described, supra, once a NOD mouse in any treatment group became diabetic, the normal treatment was stopped and was replaced immediately with 60 mg/kg GABA. Diabetes was diagnosed in these mice anywhere from 17 to 44 weeks of age. At about 32 weeks of age, approximately 50% of the mice in each treatment group became diabetic and were then treated with 60 mg/kg GABA.

### Example 4

Female non-obese diabetic (NOD) mice (Jackson Laboratory) were used to study GABA treatment efficacy. Diabetes normally occurs in 90-100% of these mice by 30 weeks and they develop the insulitis and hypoinsulinemia that characterizes Type 1 diabetes in humans. Mice were housed in accordance with NIH Animal Use and Welfare guidelines with 12 hour light-dark cycle beginning at 8 weeks of age. Animals were fed a standard rodent diet ad lib overnight followed by removal of food from the cage for an 8 hour fast. Before and after the eight hour fast, blood glucose was measured weekly using a Freestyle Glucometer and then daily once mice were determined to be diabetic. Mice were considered diabetic if at the end of the 8 hours fast, on three consecutive days, glucose measurements were ≧200 mg/dl. Onset of diabetes usually began to occur when the mice were 12 weeks of age. Once diabetic, all mice received insulin subcutaneously twice daily to bring glucose to approximately 150 mg/dl and all mice but one were started on GABA treatment at 60 mg/kg twice a day (BID).

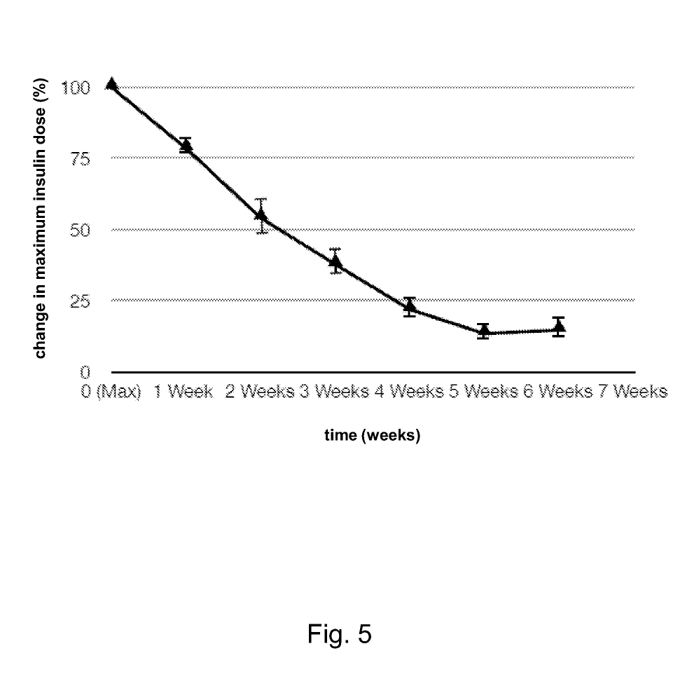

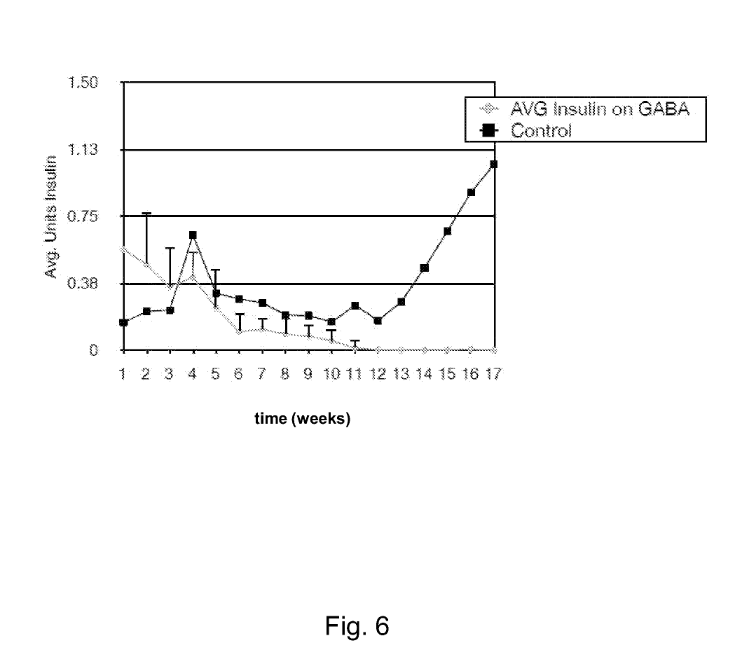

FIG. 5 shows that female NOD mice treated with 60 mg/kg GABA BID, rapidly required less insulin to keep average fasting blood glucose ≦150 mg/dl. The eight diabetic mice decreased their average insulin requirements by 85% of the maximum dose. The yellow line in FIG. 6 shows the mean insulin requirement from three of the eight mice treated with GABA. The insulin requirement of these mice to maintain their fasting blood glucose to ≦150 mg/dl eventually came to zero.

The data obtained indicate that GABA treatment in NOD mice greatly reduces exogenous insulin requirements and in some cases eliminates the need for insulin injections. All of the mice that became diabetic and were treated with 60 mg/kg GABA have experienced a ≧85% reduction in their insulin dose to keep their fasting blood glucose ≦150 mg/dl.

### Example 5

A clinical trial was conducted to study the use of oral GABA treatment in 10 naturally-occurring insulin-dependent dogs. These companion dogs were housed in homes, mimicking the lifestyle of diabetic humans. Based on published estimates, approximately 50% of these animals had insulitis, while others have had pancreatitis. It is thought that dogs do not develop type 2 diabetes and are more likely to have features of type 1.

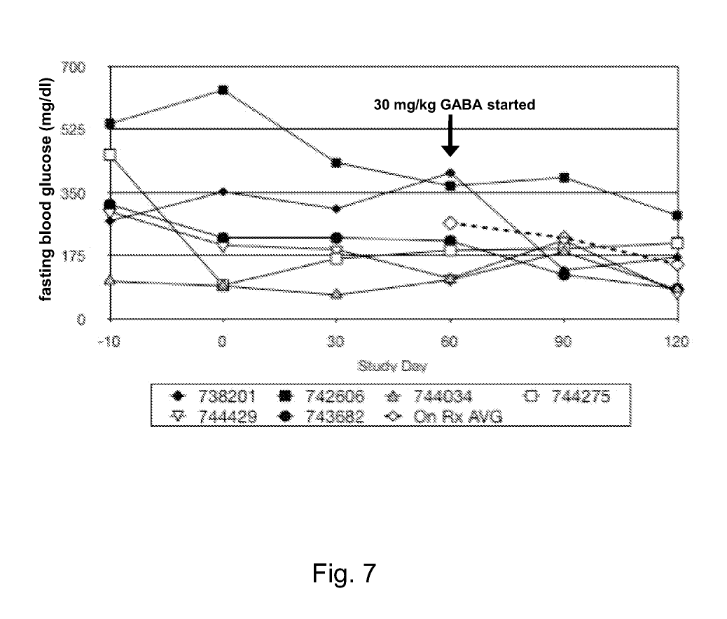

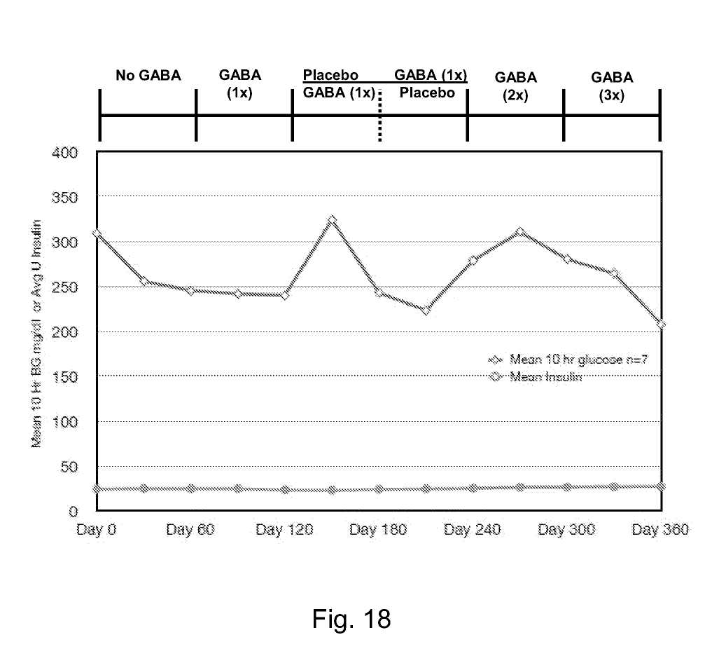

Dogs accepted into the study first began with two control months to obtain baseline glucose curves and to adjust insulin as needed to try to maintain a fasting blood glucose <200 mg/dl and/or a fructosamine <500 μmol/L. GABA treatment started on day 60, using 30 mg/kg. The GABA was diluted in water and the owner administers it orally (PO QD) as a bolus just before feeding. The dogs were kept on 30 mg/kg GABA treatment until day 120 when they are randomized into a placebo group or a group which continues the 30 mg/kg GABA treatment. Once randomized on day 120, all investigators were blinded as to treatment. The study continues for eight months with dose escalation of GABA, as described infra in Example 8 and shown graphically in FIG. 18. The data in FIG. 7 illustrates the average fasting blood glucose of six dogs that have gone through the two control months (adjusting insulin) and two months on 30 mg/kg GABA PO QD. FIG. 7 also illustrates the average fasting blood glucose in all treatment dogs, once 30 mg/kg GABA was initialed (represented by “On RX AVG” curve). Among the 6 dogs, there was a downward decrease in fasting blood glucose.

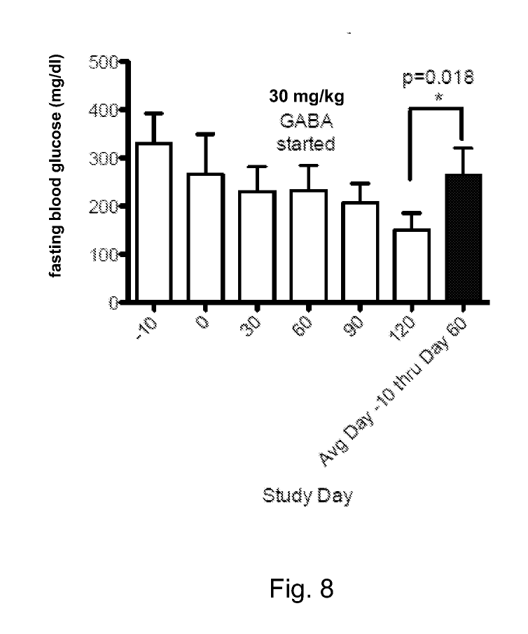

Pre-treatment average plasma glucose levels were in excess of about 264 mg/dl. Control of fasting blood glucose was substantially improved by treatment days 90 and 120. There has been a 32% average reduction in blood glucose levels from day 60 to day 120. Also, there has been a 40% reduction in average blood glucose levels at day 120 compared to the pre-treatment average from day −10 through day 60. Days −10 to 0 represent the time before the dogs were accepted into the study. Further, as shown in FIG. 7, all six dogs had reductions (ranging from −7% to −64%) in fasting blood glucose levels by day 120 compared to their pre-treatment day −10 through day 60 averages. Paired t-tests comparing day 120 FBG levels to the mean of days 10 through 60 are statistically significant as shown in FIG. 8 (p=0.018).

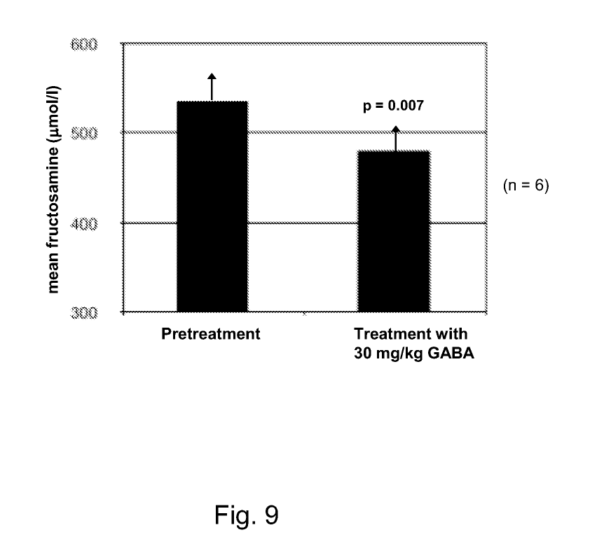

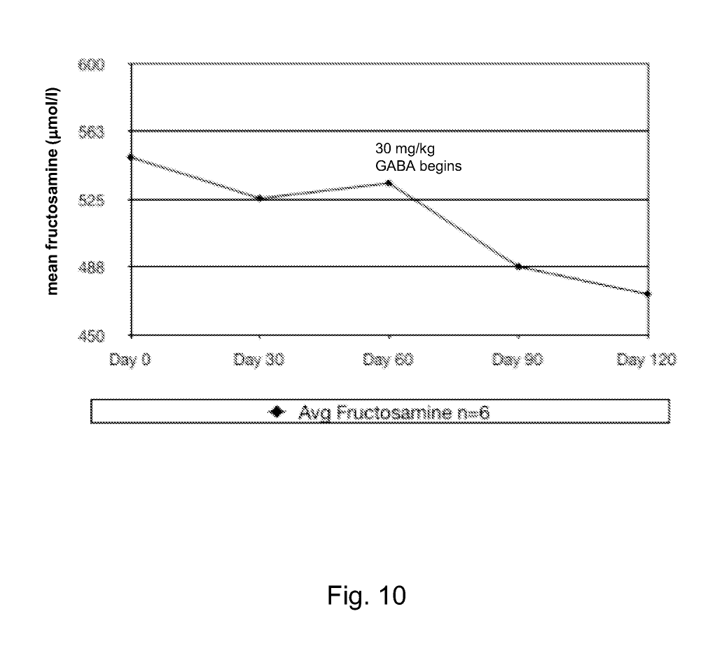

FIGS. 9 and 10 show the mean fructosamine level (μmol/l) in six dogs. In this study, a fructosamine level <500 μmol/l was considered to be good glycemic control of diabetes. The average fructosamine was significantly higher during the pretreatment (days 0-59) compared to the level during the treatment period (days 60-120) using 30 mg/kg GABA (p=0.007). FIG. 10 shows the decline of the mean fructosamine level once treatment with 30 mg/kg GABA began on day 60.

The completion of this dog study, from treatment months 3 to 8, is described in Example 8, infra.

### Example 6

Female NOD mice (NOD/ShiLtJ, Taconic) were housed in accordance with NIH Animal Use and Welfare guidelines with 12 hour light-dark cycle beginning at 9 weeks of age. The animals were fed a standard rodent diet ad lib. Blood glucose was measured weekly using a Freestyle glucometer, and then twice daily once it was determined they had diabetes.

Mice were considered diabetic if a single non-fasting blood glucose measurement was ≦200 mg/dl. For about eight weeks before the mice became diabetic, their average fasting blood glucose was 102 mg/dl. Once diabetic, blood glucose levels were measured twice daily, approximately eight hours apart. All of the mice received insulin subcutaneously twice daily and the dosage was adjusted as needed. Three diabetic mice were randomly designated as controls and were given the amino acid lysine (8 mg/kg) orally (PO) twice a day before the administration of insulin. The other NOD mice (n=5) were treated with 60 or 90 mg/kg GABA, in lieu of lysine, but otherwise were treated identically to the control group.

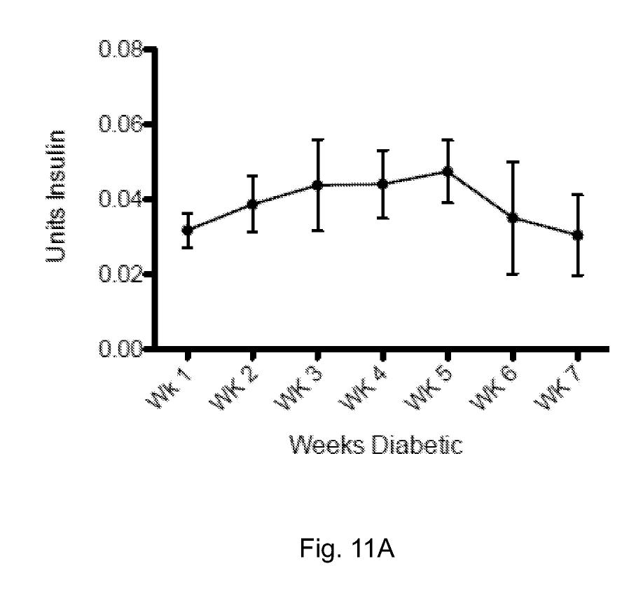

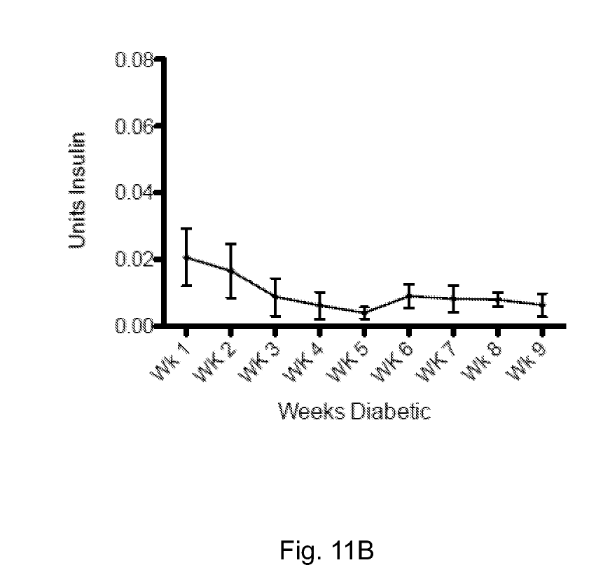

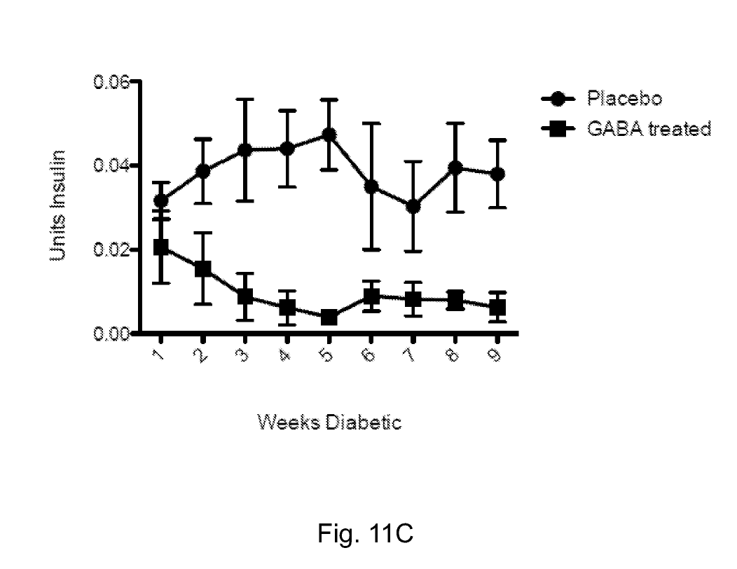

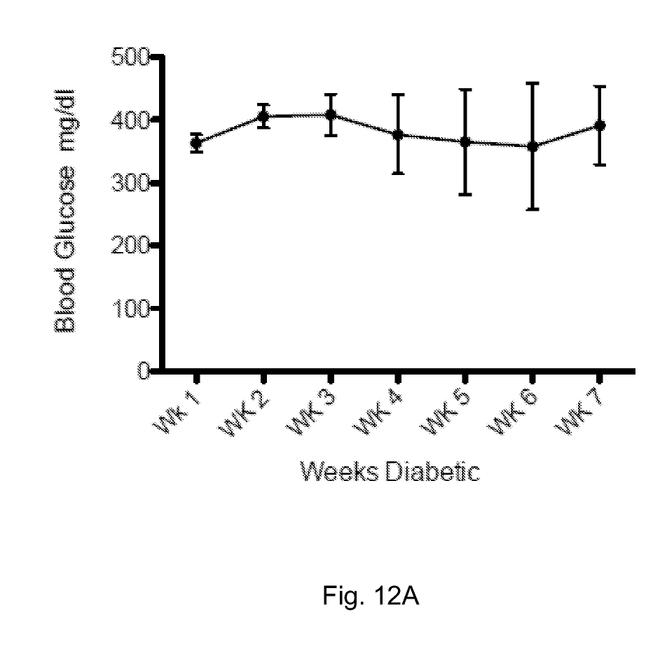

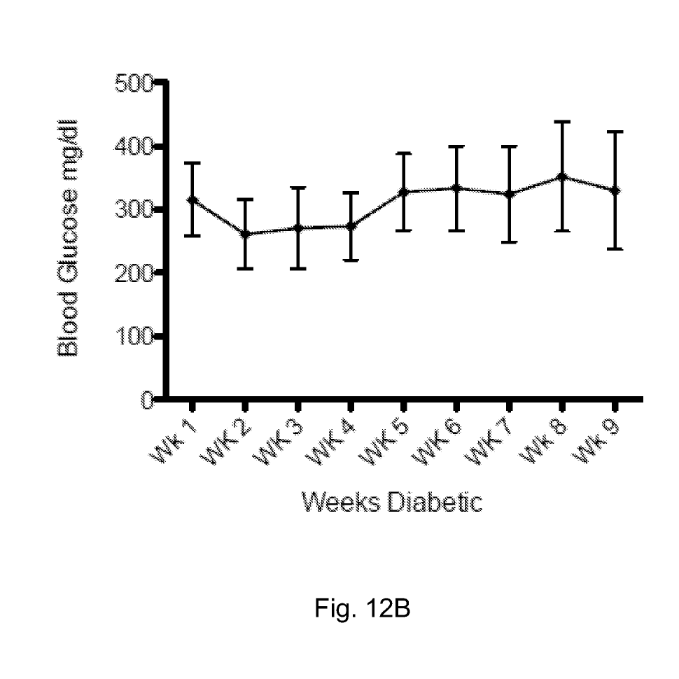

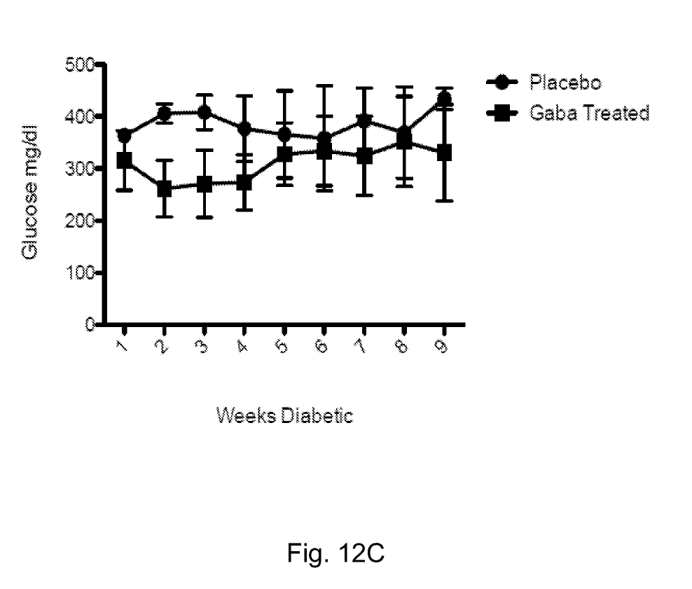

The insulin requirements in the control group, treated with lysine, ranged from 0.030-0.047 units/day over the 9 week study period, shown in FIG. 11A. In the diabetic mice treated with GABA, the required insulin dose ranged from 0.004 to 0.020 units/day, shown in FIG. 11B. FIG. 11C is the data from FIGS. 11A and B on a single graph. During the 9 week study period, the control mice had a blood sugar ranging from 362 to 463 mg/dl, seen in FIG. 12A. The blood glucose ranged from 261 to 352 mg/dl in the GABA treated mice, seen in FIG. 12B. FIG. 12C is the data from FIGS. 12A and B on a single graph. In the GABA treated group, 50% of the mice (4 out of 8) became insulin independent, no longer requiring daily insulin.

The NOD mice treated with 60 or 90 mg/kg GABA had a 4 fold decrease in insulin compared to control mice to have a mean blood glucose of 310 mg/dl over 9 weeks. The NOD mice not treated with GABA had a mean blood glucose of 386 mg/dl. In the GABA treated group, 50% of the mice did not require insulin and maintained a blood glucose average below 150 mg/dl on oral 60 or 90 mg/dl GABA alone.

None of these mice prematurely died during the course of this experiment.

### Example 7

Pancreas Histology

NOD mice, as previously described in Examples 1-4, supra, were anesthetized with an intraperitoneal injection of 75 mg/kg pentobarbital (Nembutol). When mice were unresponsive to painful stimuli, the pancreas was surgically removed and placed in 10% neutral buffered formalin. Mice were then euthanized by exsanguination. The paraffin blocks of fixed pancreas were processed by automation and 5 micron sections were prepared from three levels for automated hematoxylin and eosin staining followed by aldehyde fuchsin stain for insulin. The histology slides were prepared and stained at Jackson Laboratory, Bar Harbor, Me.

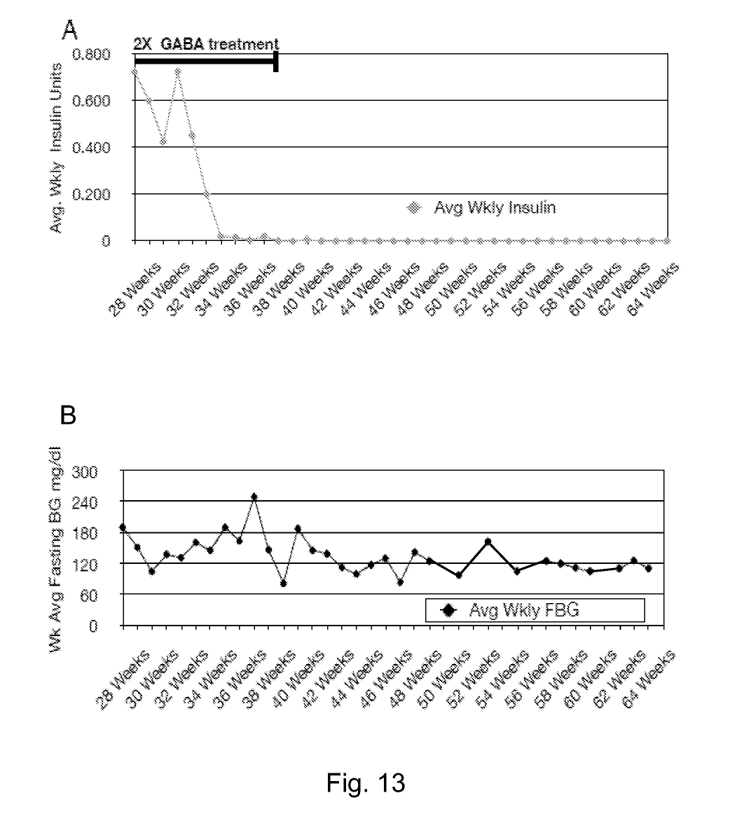

The results shown in FIGS. 13A and 13B were from a single NOD mouse, which was representative of the four mice weaned off insulin. FIG. 13A shows the average weekly insulin dose for that mouse and the duration of the 2×GABA treatment period. FIG. 13B is a plot of the average weekly fasting blood glucose (mg/dl) in the same mouse.

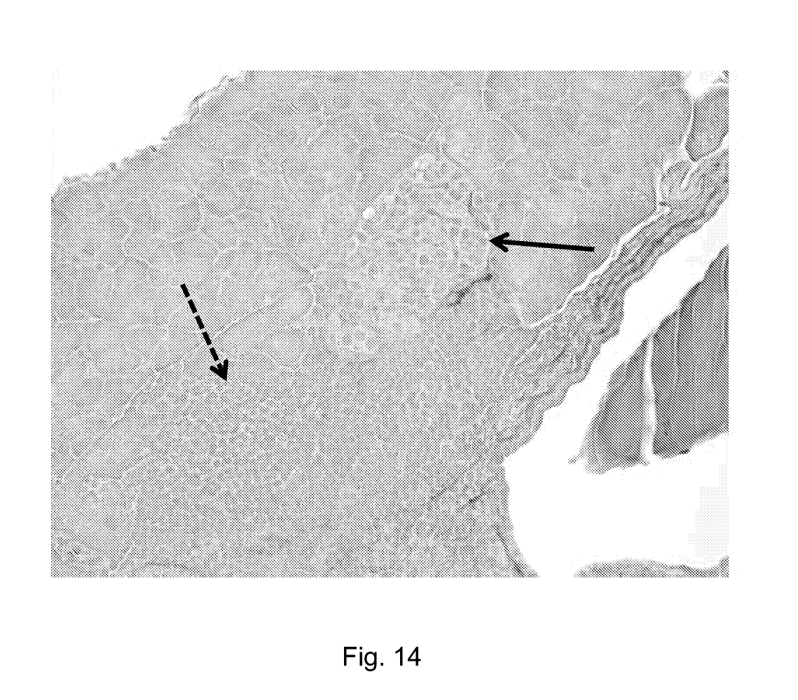

At 64 weeks, the mouse in FIG. 13 was sacrificed for pancreatic histology, shown in FIG. 14. In FIG. 14, this mouse's pancreas shows an immune reaction (dotted arrow) approaching a large islet (solid arrow), suggesting incipient diabetes, despite a normal blood glucose. Despite the return to insulin independence, the histology in FIG. 14 indicates that stopping GABA treatment for these mice causes the inflammation to return to the pancreas.

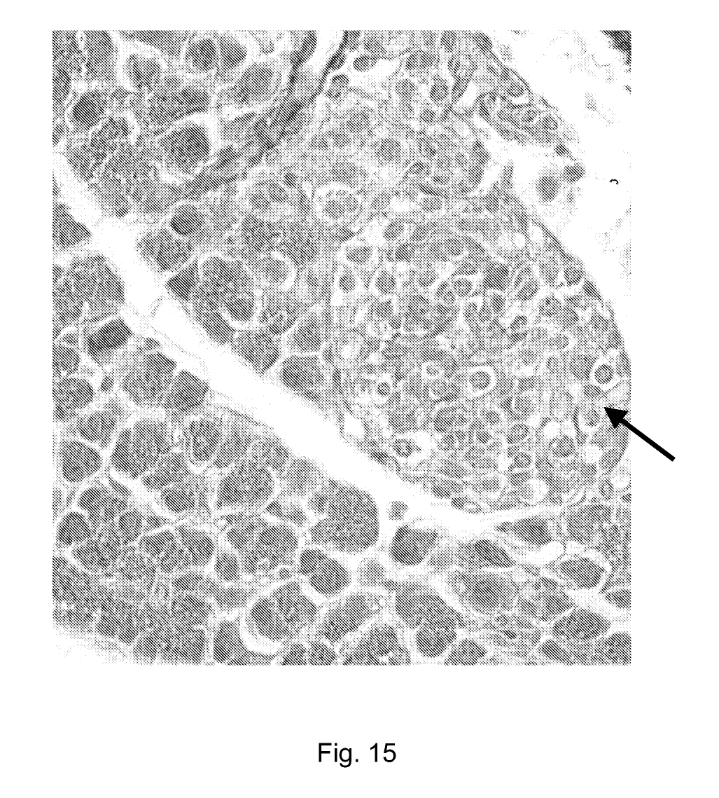

In contrast, FIG. 15 shows that a NOD mouse that remained on GABA throughout the study with regenerated islets and little to no inflammation. FIG. 15 also shows pancreatic histology of a representative mouse that remained on GABA treatment throughout the study period until the time of sacrifice, at about 60 weeks of age. The arrow points to a robust islet without surrounding inflammation. This mouse was diabetic for 34 weeks and was treated with both insulin and GABA throughout that period. Although the insulin dose at sacrifice was less than 1% of the original dose, the mouse required 0.0025 units to maintain an average weekly blood glucose of 200 mg/dl. GABA treatment in this mouse reduced insulin dependence and reversed both glucose abnormalities and islet inflammatory infiltrate as shown on histology sections.

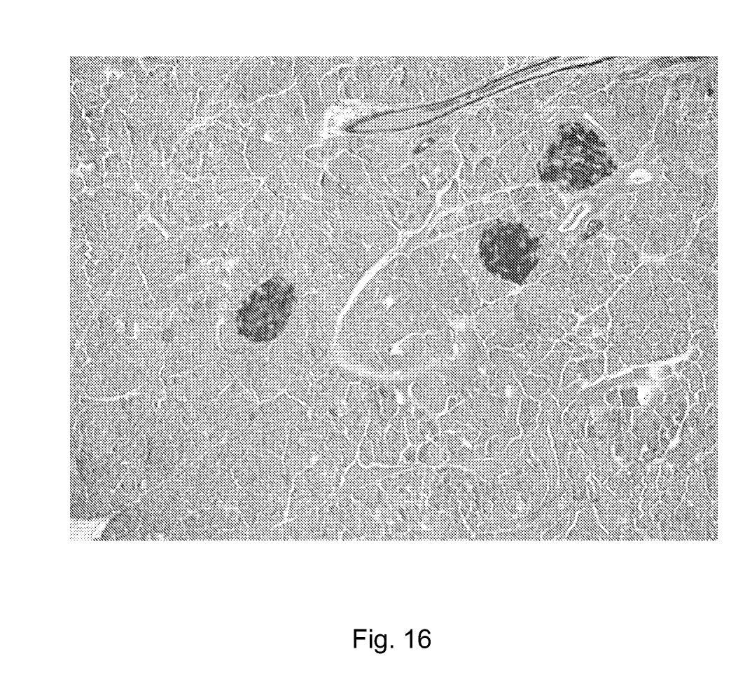

Similarly, FIG. 16 shows histology from a normal, non-NOD mouse pancreas stained with aldehyde fuchsin for insulin. Pancreatic islets, stained dark purple, are normal in size and shape, and in number.

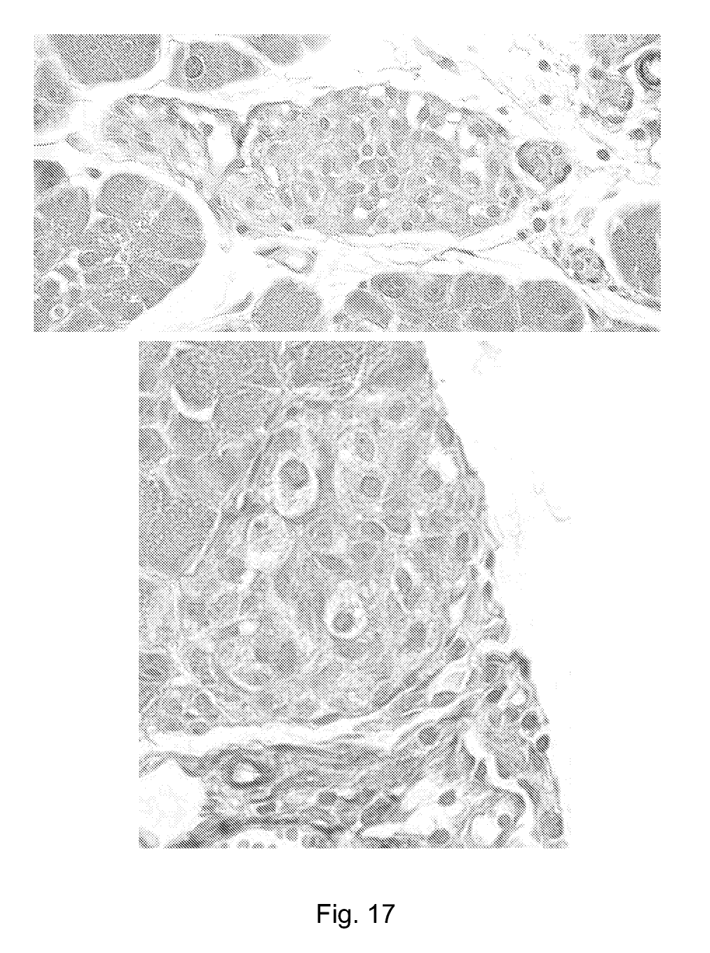

The two panels in FIG. 17 show histology from a control NOD mouse pancreas, untreated with GABA. Unlike the islets from a normal, non-NOD mouse, the islets are disorganized with vacuoles. Most of the beta cells have been destroyed or have died.

### Example 8

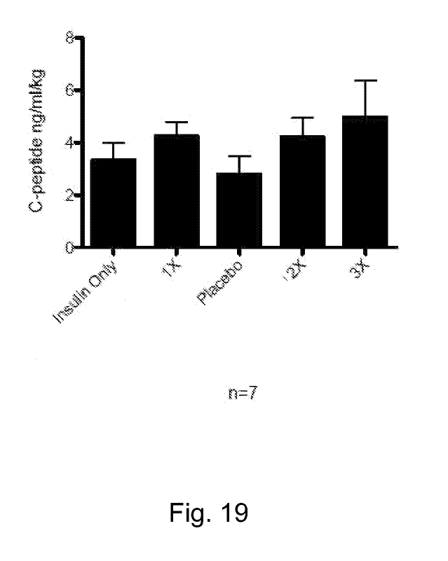

As previously described in Example 5, supra, the 8 month clinical canine trial in collaboration with Purdue University was completed. Naturally-occurring diabetic dogs were housed in homes and were insulin dependent when they began the study. FIGS. 18 and 19 show the mean 10 hour fasting blood glucose (mg/dl) and mean C-peptide (ng/ml/kg) in these insulin-dependent dogs, respectively.

Fasting Blood Glucose Measurements

FIG. 18 is a graph of the mean blood glucose in the seven dogs measured during a ten hour window. On a measurement day, the dogs were fed and given insulin. Blood glucose was then monitored at ˜8:00 AM and for every two hours until ˜6:00 PM. The mean of these values was taken and plotted in FIG. 18.

On day 0, the mean 10 hr glucose was 310 mg/dl. From day 0 to day 60, the dogs were treated with insulin only. Starting on day 60, GABA was given orally at a dose of 30 mg/kg. On day 120, 3 out of 7 dogs were placed on placebo for two months and then at day 180 went back on 30 mg/kg GABA until day 240. The other four dogs, received the reverse treatment: they received 30 mg/kg GABA between days 120-180 and then were started on placebo until day 240. All seven dogs received 60 mg/kg GABA that was started on day 240. The GABA dose increased to 90 mg/kg on day 300 in all dogs and remained at that dose until the end of the study. The mean blood glucose over the 10 hour measurement period dropped to 209 mg/dl by the last visit, a 33% overall improvement without a significant change in the average insulin dosage.

C-Peptide Measurements

C-peptide was monitored as a way to determine the amount of endogenous insulin synthesized and secreted in these diabetic dogs. Endogenous insulin is first biosynthesized as linked A, B and C peptides. The C-peptide is removed and circulates in the blood stream. The active insulin molecule is linked A & B peptide only. Since diabetic patients administering insulin injections receive exogenous A & B peptide, the measurement of C-peptide can determine of how much endogenous insulin is produced by the individual's pancreas.

In this study stimulated C-peptide was measured every two months. On the day of C-peptide measurements, blood was drawn at time 0 and the dog was fed stimulating C-peptide release. No exogenous insulin was given by injection to avoid suppressing endogenous insulin secretion. Blood was drawn again at 60, 90, 120 and 240 minutes. The graph in FIG. 19 represents the mean C-peptide, measured by radioimmunoassay, during the 1× (30 mg/kg), 2× (60 mg/kg), and 3× (90 mg/kg) GABA treatment, for the seven dogs. C-peptide was plotted as area under the curve measurements. The values for the placebo treatment have been grouped and represent all seven dogs.

This study showed a dose dependent increase in C-peptide when GABA was administered orally to insulin dependent dogs. The level of C-peptide decreased during the placebo phase, but increases with continued GABA treatment. The data indicates that pancreatic beta cell function was maintained and may improve at higher doses of GABA.

Pharmacokinetics

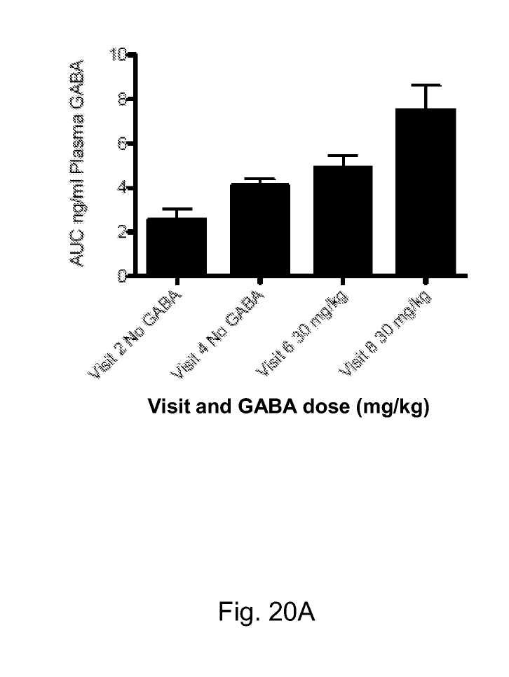

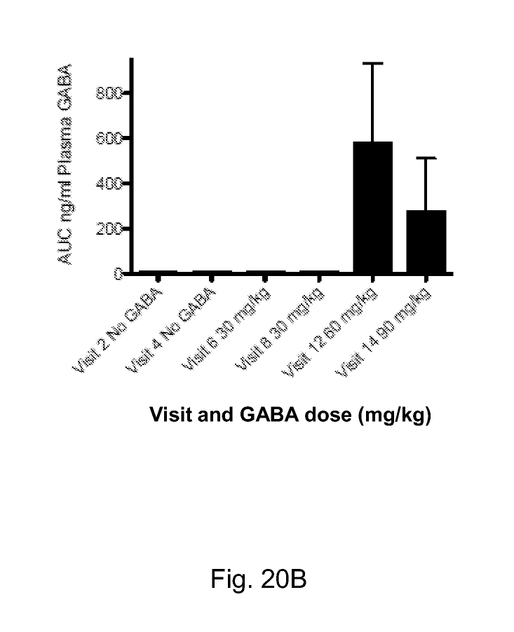

FIGS. 20A and B represent the mean plasma concentration of GABA (ng/ml) at different doses of oral GABA administered to diabetic, insulin-dependent companion dogs. Plasma GABA measurements were made by liquid chromatography/mass spectroscopy. Blood was drawn at 0, 60, 90, 120 and 240 minutes and plotted as area under the curve. These samples were drawn after a meal but in the absence of exogenous insulin administration, as previously described for C-peptide measurements. Blood samples were drawn and divided into serum separation or plasma separation tubes for C-peptide and pharmacokinetics studies respectively.

In this study, GABA was not administered before blood was drawn during visits 2 and 4, so these levels reflect endogenous plasma GABA levels. FIG. 20A shows an increase in plasma GABA between visit 2 and 4, and may have been due to an improvement in overall health, since the blood glucose was under better control with insulin adjustments by the veterinarian, shown in FIG. 18, days 0 to 60. GABA levels continued to rise as oral administration of GABA began on visit 6 and escalated over subsequent visits. The plasma GABA levels increased almost 80 fold between visit 8 and visit 12 when the GABA dose was doubled but decreases two fold when the dose was raised to 90 mg/kg at visit 14 (FIG. 20B). This bell shaped response mimics what is seen in dose-response curves with GABA in vitro.

The circulating level of GABA in the control dog was greater than the mean plasma GABA level in the diabetic dogs, before GABA treatment (data not shown). This suggests that the primary source of circulating GABA is from pancreatic beta cells, which are depleted in diabetic dogs. Circulating GABA may also be diminished before the onset of diabetes and could be used as a diagnostic tool to predict those at risk for type 1 diabetes.

The relevant teachings of all patents, published applications and references cited herein are incorporated by reference in their entirety.

While this invention has been particularly shown and described with references to example embodiments thereof, it will be understood by those skilled in the art that various changes in form and details can be made therein without departing from the scope of the invention encompassed by the appended claims.

What is claimed:

1. A method of delaying the onset of diabetes in a mammal subject to developing diabetes comprising:
periodically administering to said mammal an effective diabetes-delaying amount of a GABA receptor agonist at or near ingestion of meals by said mammal.

2. The method of claim 1, wherein the GABA receptor agonist comprises GABA.

3. The method of claim 2, wherein the mammal is a human or a canine.

4. The method of claim 3, wherein the effective diabetes delaying amount of the GABA receptor agonist is an amount from about 30 mg/kg to about 120 mg/kg.

5. A method of reducing the insulin dosage in a mammal being treated for diabetes with periodic exogenous administration of insulin comprising:
co-administering an effective insulin-reducing amount of a GABA receptor agonist at or near the periodic administration of insulin.

6. The method of claim 5, wherein the GABA receptor agonist comprises GABA.

7. The method of claim 6, wherein the mammal is a human.

8. The method of claim 7, wherein the effective insulin-reducing amount of the GABA receptor agonist comprises at least 30 mg/kg (60).

9-16. (canceled)

17. A kit comprising:
a GABA receptor agonist and insulin.

18. The kit of claim 17 wherein the GABA receptor agonist comprises GABA.
